(19) United States
(12) Patent Application Publication
(10) Pub. No.: US 2018/0256405 A1
(43) Pub. Date: Sep. 13, 2018

(54) System and Apparatus for Irrigating a Wound

(71) Applicant: Peter Yen, Birmingham, AL (US)

(72) Inventor: Peter Yen, Birmingham, AL (US)

(21) Appl. No.: 15/453,495

(22) Filed: Mar. 8, 2017

(51) Int. Cl. A61F 13/00; A61F 13/02; A61M 27/00; A61M 1/00; A61M 3/02; A61M 5/148

(52) U.S. Cl. CPC A61F 13/00068; A61F 13/0216; A61M 27/00; A61M 1/0058; A61M 3/0287; A61M 2205/3344; A61F 13/0226; A61M 1/0001; A61F 2013/00536; A61F 2013/0054; A61M 5/1486

(57) ABSTRACT

A wound irrigation system and apparatus for irrigating a wound comprising an irrigant reservoir for holding an irrigant solution, a delivery line, a flow regulator, a pump in communication with the reservoir to apply pressure to the reservoir and an irrigation attachment that can be arranged in varying configurations in a wound site to allow uniform irrigant coverage in a wound site on a patient. The irrigation attachment is comprised of a trunk line and branch lines that are in fluid communication with the trunk line and whereby said branch lines can be manipulated into position by a healthcare provider into the appropriate areas of a wound site.

### CROSS-REFERENCE TO RELATED APPLICATIONS

None

### FEDERALLY SPONSORED RESEARCH

None

### SEQUENCE LISTING

None

### FIELD OF THE INVENTION

The invention relates to a wound irrigation drip system and apparatus and method that allows for a wound to be automatically irrigated for a period of time with the appropriate irrigant fluids. The invention allows for the predictable application of a wound irrigant into a wound and associated dressing with minimal supervision and time required by a medical/healthcare provider or clinician.

### BACKGROUND OF THE INVENTION

Patient wounds often require a steady application of antibacterial and/or antiseptic irrigants to the wound site to avoid infection and to also keep a wound sight from scabbing up prematurely. These wounds irrigants are further used to treat and decontaminate the wound or resolve a local infection at the wound site prior to definitive surgical closure via suture closure or skin grafting. Further, wounds that are not candidates for surgical intervention can be treated by the use of wound antibacterial and/or antiseptic irrigants. This requires a clinician to routinely soak a wound and the wound dressing applied thereto a number of times a day.

The problem with treating wounds through the use of irrigants is that it requires frequent attention by a healthcare provider to monitor the wound and wound dressing. Often times an irrigant needs to be applied every one to six hours. Further, it must be applied manually by a healthcare provider who may have too many patients, among other issues, to consistently administer care on a desirable frequency. If a wound irrigant is not applied frequently enough to a wound site there are a number of deleterious effects that can and will result. One negative effect is that any infection present in the wound site will not be treated and could potentially fester and become worse. This can lead to prolonged recovery in advance of surgery, prolonged hospital stays, and possible irreversible damage to the wound site. Further, inadequate irrigant treatment could lead to an infection that otherwise would not have occurred. Another negative effect is that gauze dressing that is directly adjacent and in contact with a wound site will dry out and cause the wound site to stick to the gauze dressing. This requires a healthcare provider to dislodge the wound dressing from a wound site causing pain, bleeding, local trauma during dressing removal, disruption of healing, increased wound depth and size, and further injury and disruption of the healing process by opening wounds back up from a previously achieved healing progress. Additionally, if a wound is over saturated, it can cause skin maceration which is the softening and breaking down of skin from prolonged exposure to moisture which can lead to the increase of the size of the wound. These negative effects are directly related to a healthcare provider's inability to provide the consistent and frequent wound care that some wounds require.

While there are systems and apparatus that exist in the prior art that attempt to address the problem of delivering automated irrigation to wounds, they are expensive and overly complex in use to be commercially and/or medically viable. What is needed in the art is an automated wound irrigation system and apparatus and method that is relatively inexpensive, predictable, effective and easy for a healthcare provider to use and administer. The present system and apparatus and method allows for sterile wound irrigation that can be customized to accommodate any wound size and body area that is able to be incorporated into any types of irrigant dressings.

The present invention and method further provides continuous irrigation and a customizable irrigant saturation level, duration and frequency. The present invention and method is further self-contained such that it only requires addition of medicinal irrigant. The present system can also be used in under sterile conditions such as that found in an operating room, it can be adjusted or customized at any time by the healthcare provider to provide the desired effect and it is reusable for the same patient. Indirectly, the benefits provided by the present invention and method include: reduced workload of healthcare worker to saturate a patient dressing, elimination of the risk of complications associated with under or over saturation of dressing, reduction in human error, inspiring confidence on proper wound saturation for an entire day, ensures quality patient care while reducing workload, lowers overall healthcare costs due to reduced healthcare provider workload and time required to run invention, and safe for a healthcare provider to use with minimal training.

### SUMMARY OF THE INVENTION

The present invention comprises a irrigant reservoir that is attached to a delivery line that is further attached to an irrigation attachment device. The reservoir has an inlet and an inlet cap for loading an irrigant solution, an air inlet and air inlet line which allows the reservoir to be pressurized by using a pump connected to said air inlet line and a pressure gauge attached to the air inlet line to indicate pressure to a user. The reservoir has an outlet and an outlet line with a flow regulator located on said outlet line. The reservoir outlet line connects to the delivery line by way of commonly used male and female catheter connectors. The irrigant attachment has a trunk line with a connector located on its upper end. The trunk line has branch lines that are formed in fluid communication with the trunk line such that the branch lines are formed into the sidewall of the trunk line so that fluid can pass from the trunk line into the branch lines. The branch lines can either be formed into the sidewall of the trunk line at a central position or in staggered positions along the trunk line. The branch lines can be cut to correspond to a particular wound area for advantageous placement.

Specific advantages and features of the present invention will be apparent from the accompanying drawings and the description of several illustrative embodiments of the present invention.

### DESCRIPTION OF THE DRAWINGS

The invention is generally depicted in FIGS. 1-6 but may be embodied in various other forms. The principles and teachings of the invention, therefore, can be applied to numerous alternative variations.

FIG. 1 is a perspective view of a wound irrigation system and irrigation attachment.

FIG. 2. is a perspective view of an alternate embodiment of an irrigation attachment.

FIG. 3 is a perspective view of alternate embodiment of a wound irrigation attachment.

FIG. 4 is a perspective view of a manifold for use in in a wound irrigation attachment.

FIG. 5 is a cut away side view of the manifold.

FIG. 6 is a perspective view of an irrigation attachment installed onto a wound site of a patient.

### DETAILED DESCRIPTION

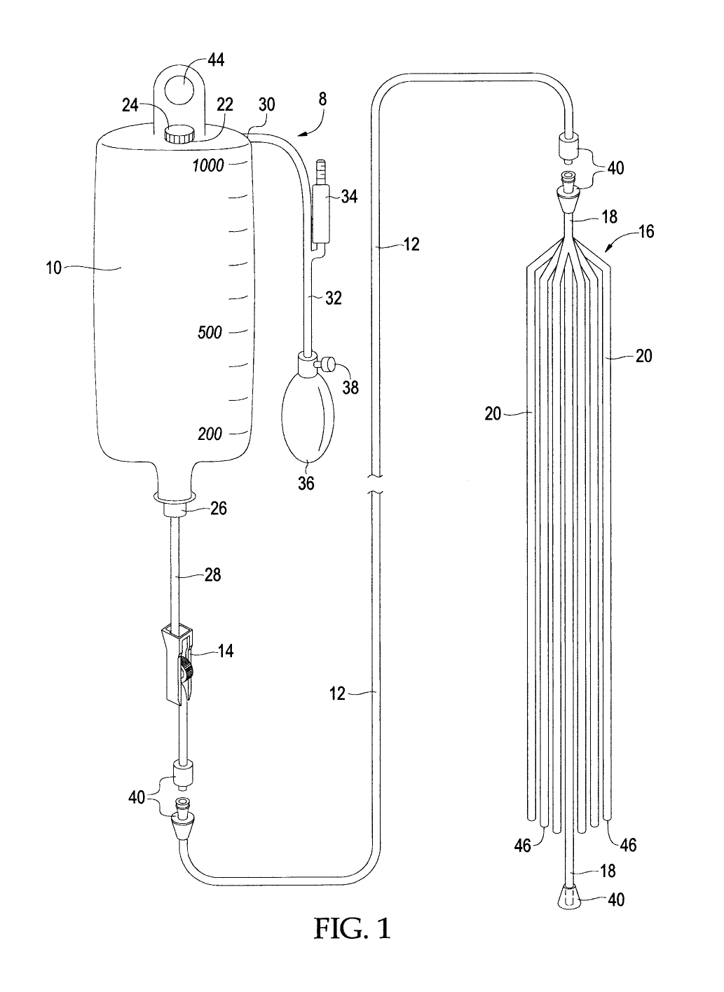

Referring now to FIG. 1, there is shown a wound irrigation system 8 having a reservoir 10, a delivery line 12 and an irrigant attachment 16. The reservoir 8 is formed of materials common to reservoirs formed for the administration of intravenous fluids such as polyvinyl chloride, ethylene vinyl acetate, polypropylene, copolyester ether. The reservoir 10 has an inlet 22 with a sealable inlet cap 24. The reservoir 10 also has an air inlet 30 that is in fluid communication with the reservoir by way of the air inlet line 32. The air inlet line 32 has at its terminus a pump 36 for pressurizing the reservoir 10 as well as a pressure gauge 34 that enables a healthcare provider to set the appropriate pressure. The reservoir also has means for suspending 44 the reservoir 10. One means for suspension 44 is is a handle or loop formed in the top portion of the reservoir 10 out of the same material of which the reservoir 10 is made. Another means for suspending the reservoir 10 is to form points of attachment such as apertures in the upper surface of the reservoir 10 such that a handle made of plastic, metal or wood or other rigid material can be installed into the points of attachment. This allows the reservoir 10 to be suspended by hanging it on a suitable stand that has a hook or other fixed hanging point such that the reservoir 10 is secure and placed above a patient such that the reservoir 10 takes advantage of the gravity. The reservoir 10 also has volumetric gradations located on its outer surface for purposes of indicating and measuring the amount of irrigant that is poured into the reservoir 10.

The pump 36 can be of the variety that are commonly found on traditional blood pressure cuffs where the pump 36 is a bulb shaped device that is mechanically actuated by a healthcare provider's hand and has a stopcock 38 to lock in the pressure once the pump 36 has been actuated to reach a desired reservoir 10 pressure. In an alternate embodiment, the pump 36 can be an electric air pump that will allow the reservoir 10 to maintain a constant desired pressure through use of a microprocessor, pressure sensors and pre-programmed algorithms for maintaining pressure.

Further shown in FIG. 1, the reservoir 10 has an outlet 26 and an outlet line 28. Along the outlet line 28 length is located a flow regulator 14. The flow regulator 14 can be a variety of different types including a roll clamp, a dial flow, or a stopcock flow regulator among other various types of intravenous line and/or tubing flow regulators 14. The outlet line 28 terminates into a connector 40. The connector 40 can be of any of a variety of connecting and/or coupling devices uses on plastic tubing and/or intravenous lines. There is also shown a delivery line 12 that connects to the outlet line 28 and to the irrigation attachment 16 via connectors 40. The irrigation attachment 16 shown in FIG. 1 has a trunk line 18 and branch lines 20. The delivery line 12, outlet line 28, trunk line 18 and branch lines 20 can be made of a variety of materials including polyvinyl chloride, polyethylene, polyurethane, rubber and a variety of other suitable materials. In the embodiment shown, the trunk line 18 has branch lines 20 that are formed into the sidewall of the trunk line 20 at an upper region on the trunk line 18. The trunk line 18 extends down below where the branch lines 20 are joined to the trunk line 18 such that it extends a distance past the branch lines 20 and terminates into a connector 40. The connector 40 is present to allow connection to another irrigation attachment 16 in series such that multiple irrigation attachments 16 can be used at once. The use of multiple irrigation attachments 16 in series would be helpful in the case where the wound area is large. In one embodiment, the branch lines 20 are smaller in diameter than the trunk line 18 such that the smaller diameter branch lines 20 act to restrict overflow of the irrigant and allow for uniform irrigation. The branch lines 20 have open ends 46 for the delivery of an irrigant.

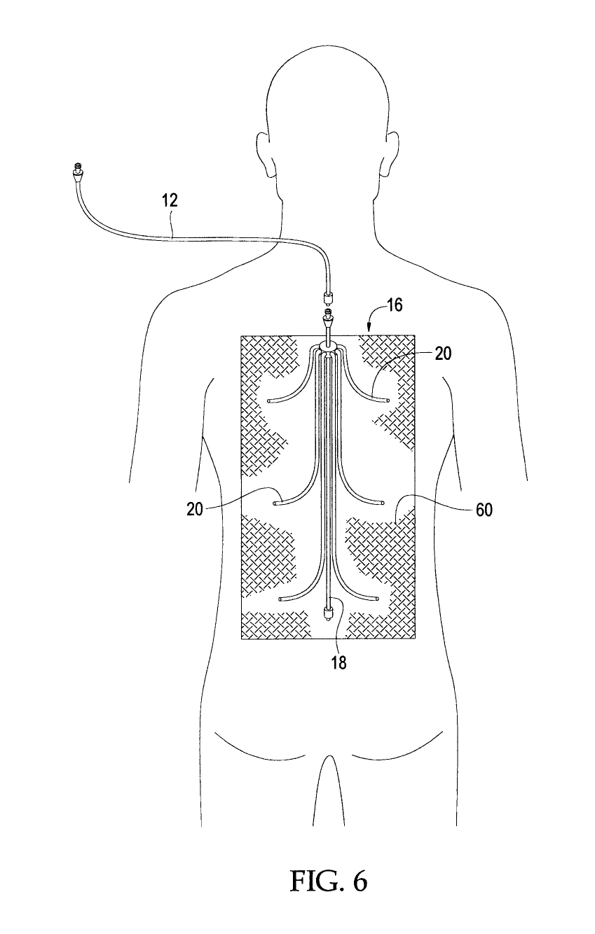

Referring again to FIG. 1, the irrigation attachment can be modified and customized to fit any wound by trimming the branch lines 20 to a desired length and placing them in a wound area such that sufficient irrigant coverage is provided. In addition, the branch lines 20 can be perforated on their sidewalls either in the manufacturing process or by a healthcare provider to enhance the irrigant coverage in the wound. In normal circumstances, gauze or other type cloth dressing 60 (see FIG. 6) is used to place over the wound. The branch lines 20 can be taped to the underside of the dressing 60 in various positions to give optimal coverage for irrigation of the wound without fear that the branch lines 20 will move out of position. The branch lines 20 and the trunk line 18 in one embodiment can be coated with a silver ion coating to prevent the spread of harmful bacteria and microbes in the wound.

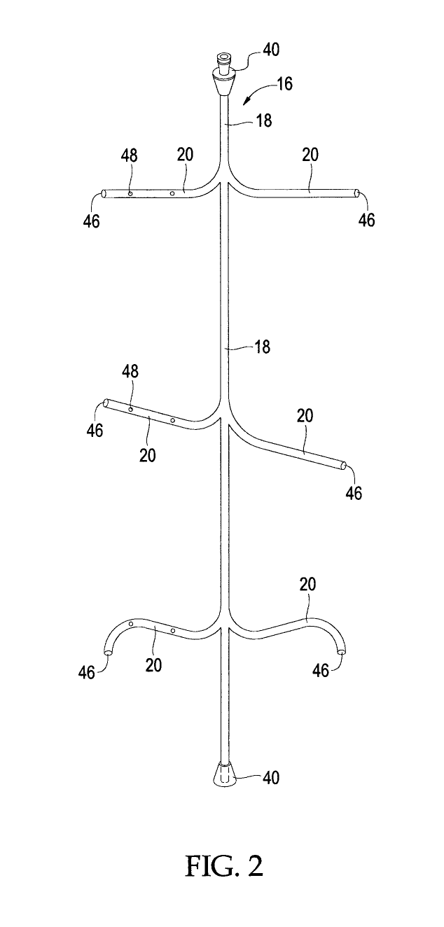

Referring now to FIG. 2, there is shown an alternate embodiment of the irrigation attachment 16. In this embodiment, the irrigation attachment 16 has a trunk line 18 and branch lines 20 whereby the branch lines 20 are joined to the trunk line 18 at different heights or in a staggered formation. The branch lines 20 are formed in direct communication with the trunk line 18 such that fluid can flow from the trunk line 18 into the branch lines 20. The irrigation attachment 16 can be made by injection molding or tubing extrusion type processes or a combination of both. The irrigation attachment 16 has a connector 40 on the top end of the trunk line 18 and a connector 40 on the bottom end of the trunk line 18. The connector 40 on the bottom end of the trunk line 18 is used to add an additional irrigation attachment 16 such that multiple irrigation attachments 16 can be chained together in series for larger wounds. Also shown are apertures 48 located along the length of the branch lines 20. These apertures 48 allow for more even coverage and introduction of irrigant in a wound. The apertures 48 can be either formed into the branch lines 20 during the manufacturing process or they can be custom added later as dictated by a particular wound by a health care professional by using a blade, punch or other sharp instrument to form the aperture 48 in the branch lines 20.

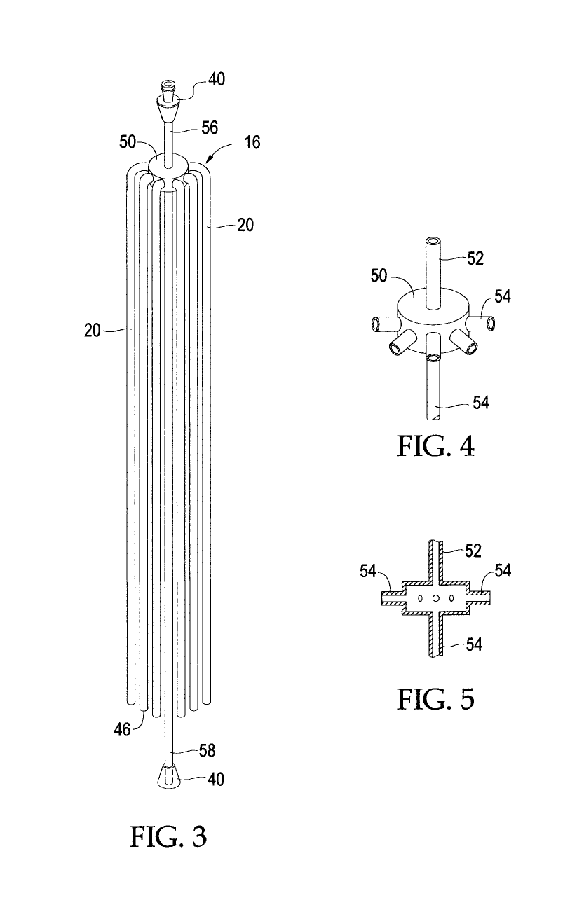

Referring now to FIGS. 3-5, there is shown an alternate embodiment of the irrigation attachment 16 whereby a manifold 50 serves as a junction between a first trunk line 52, branch lines 20 and a second trunk line 58. The manifold 50 has a top nipple 52 and plurality of nipples 54 located on its outer surface. The manifold 50 has a hollow chamber such that the top nipple 52 is in fluid communication with the nipples 54 located on the outer surface of the manifold 50 by means of hollow passageways in the top nipple 52 and nipples 54. In the embodiment shown, the manifold 50 is of a cylindrical shape, however, the manifold 50 could be made of any variety of shapes to achieve the same result of transferring fluid therethrough. Other shapes that could be employed include a horizontal linear tube, a frusto-conical body, a tree-shaped manifold 50 such that there is a main trunk tube with branching tubes that correspond to the first trunk line 56, branch lines 20 and second trunk line 58. The irrigation attachment 16 shown in FIGS. 3-5 could either come pre-installed from the factory or a healthcare professional could install the first trunk line 56, second trunk line 58 and branch lines 20 onto the manifold 50. The irrigation attachment 16 first trunk line 56 and second trunk line 58 have a connector 40 installed onto their ends. The manifold 50 ideally would be made of a plastic material and manufactured through the process of injection molding, 3-D printing, or cast molding but other materials could work as well. The branch lines 20 have open ends 46 for the introduction of irrigant to wound and apertures can be formed along the length of the branch lines 20.

Referring now to FIG. 6, there is shown an irrigant attachment 16 installed on patient such that the branch lines 20 are located in regional positions such that uniform coverage of a wound is provided. A dressing 60 is placed over the wound and irrigant attachment 16 to maintain appropriate moisture and protection from microbes, bacteria, viruses and infection of the wound site in general. The irrigant attachment 16 can be attached the to the underside of the dressing 60 using surgical tape or other types of tape such that the branch lines 20 will maintain their position in the wound site because they are affixed to the underside of the dressing 60 and the dressing 60 is affixed to the patient using tape.

In use, a healthcare provider would suspend the reservoir 10 as shown in FIG. 1 on an appropriate stand. They would attach the delivery line 12 to the reservoir outlet line 28 and the delivery line 12 to the irrigation attachment 16. The irrigation attachment 16 would be arranged and/or trimmed to occupy a desired irrigant coverage in the wound site of a patient. A dressing 60 would be applied to the wound site with the irrigation attachment 16 in between. An appropriate irrigant volume would loaded into the reservoir 10 by means of the inlet 22 and sealed by use of the inlet cap 24. The healthcare provider will then adjust the flow regulator 14 to a closed position while the appropriate amount of pressure is applied to the reservoir 10 by use of the pump 36. Once the desired amount of pressure is introduced into the reservoir 10, the healthcare provider can then adjust the flow regulator 14 to a desired setting such that the desired amount of irrigant flow over a period of time is achieved.

The principles, embodiments, and modes of operation of the present invention have been set forth in the foregoing specification. The embodiments disclosed herein should be interpreted as illustrating the present invention and not as restricting it. The foregoing disclosure is not intended to limit the range of equivalent structure available to a person of ordinary skill in the art in any way, but rather to expand the range of equivalent structures in ways not previously contemplated. Numerous variations and changes can be made to the foregoing illustrative embodiments without departing from the scope and spirit of the present invention.

What is claimed is:

1. A wound irrigation system, comprising:
A reservoir;
A delivery line;
A flow regulator; and
An irrigant attachment having a trunk line and a plurality of branch lines.

2. The wound irrigation system of claim 1 wherein the irrigant reservoir has a reservoir inlet for loading an irrigant, an outlet, an outlet line, an air inlet connected to the reservoir by an air inlet line, a pressure gauge located on the air inlet line and a pump connected to the air inlet line to introduce air into the reservoir such that the reservoir is under pressure.

3. The wound irrigation system of claim 1 wherein the delivery line has a connector on one end that mates with a connector located on the outlet line and a connector on its other end which mates with a connector on the trunk line of the irrigant attachment.

4. The wound irrigation system of claim 1 wherein there is included means for suspending the irrigation reservoir.

5. The wound irrigation system of claim 1 wherein the flow regulator is located on the reservoir outlet line.

6. The wound irrigation system of claim 1 wherein said plurality of branch lines are formed in fluid communication with the trunk line such that the branch lines are formed into the sidewall of the trunk line so that fluid can pass from the trunk line to the branch lines.

7. The wound irrigation system of claim 1 wherein said branch lines have open ends and apertures formed in the sidewalls of said branch lines to facilitate the delivery of an irrigant to various locales in a wound area.

8. The wound irrigation system of claim 1 wherein said trunk line extends to a length equal to or greater than the length of the branch lines wherein the terminal end of the trunk line has a second connector installed thereon for receiving a second wound irrigation attachment.

9. The wound irrigation system of claim 1 wherein a silver ion coating is applied to said trunk line and branch lines.

10. The wound irrigation system of claim 1 wherein the irrigant attachment further comprises a manifold such that a first trunk line is insertable onto a top nipple on the manifold, said branch lines are inserted onto a plurality of nipples located on the manifold outer surface and a second trunk line is insertable onto a nipple located on the manifold outer surface whereby said second trunk line terminates into a connector.

11. A wound irrigation attachment for a wound irrigant reservoir, comprising;
a trunk line; and
a plurality of branch lines that are formed in fluid communication with the trunk line such that the branch lines are formed into the sidewall of the trunk line so that fluid can pass from the trunk line to the branch lines.

12. The wound irrigation attachment for an irrigant reservoir of claim 11 wherein the top end of the trunk line has a connector suitable for connection with a connector located on the end of a delivery line.

13. The wound irrigation attachment for an irrigant reservoir of claim 11 wherein the branch lines haven open ends and apertures formed in the sidewalls of said branch lines to facilitate the delivery of an irrigant to various locales in a wound area.

14. The wound irrigation attachment for an irrigant reservoir of claim 11 further comprising said branch lines that have different lengths

15. The wound irrigation attachment for an irrigant reservoir of claim 11 wherein a silver ion coating is applied to said trunk line and branch lines.

16. The wound irrigation attachment for an irrigant reservoir of claim 11 wherein the branch lines are formed onto the sidewall of the trunk line at different heights along the trunk line.

17. The wound irrigation attachment for an irrigant reservoir of claim 11 wherein said trunk line extends below where said branch lines are formed thereon and extends to a length equal to or greater than the length of the branch lines wherein the terminal end of the trunk line has a second connector device installed thereon for receiving a second wound irrigation attachment.

18. The wound irrigation attachment for an irrigant reservoir of claim 11 wherein the irrigant attachment further comprises a manifold such that a first trunk line is insertable onto a top nipple on the manifold, said branch lines are inserted onto a plurality of nipples located on the manifold outer surface and a second trunk line is insertable onto a nipple located on the manifold outer surface whereby said second trunk line terminates into a connector.

19. A method for irrigating a wound, comprising the steps of:
Providing a wound irrigation system further comprising:
A reservoir wherein the reservoir has an inlet for loading an irrigant, an outlet and an outlet line with a connector at its end, an air inlet and an air inlet line for introduction of air, a pressure gauge, and a pump for pressurizing said reservoir with air;
A delivery line having a connector at either end;
A flow regulator;
An irrigant attachment having a trunk line and a plurality of branch lines wherein said branch lines are in fluid communication with the trunk line so that irrigant can pass from the trunk line to the branch lines and a connector installed onto the top end of the trunk line and a connector installed onto the bottom end of the trunk line;
suspending the reservoir at a height above a wounded patient;
attaching one end of the delivery line to the reservoir outlet and attaching the other end of the delivery line to the irrigant attachment;
arranging said irrigant attachment trunk line and branch lines in appropriate positions on a wound to provide adequate irrigation to a wounded area on a body;
loading the reservoir with an irrigant solution;
actuating the pump to pressurize said irrigant reservoir to a desired pressure level;
adjusting the irrigant flow regulator to a preferred irrigant output; and
securely covering said wound area with a dressing.

20. The method of claim 19 further comprising the step of coating the trunk line and branch lines with a silver ion coating.

21. The method of claim 19 further comprising the step of forming apertures at strategic places along said branch lines to correspond to a wounded area on a body.

22. The method of claim 19 further comprising the step of cutting said branch lines to various lengths to correspond to a wounded area on a body.

23. The method of claim 19 wherein the step of providing a wound irrigation system further comprises an irrigant attachment having a manifold such that a first trunk line is insertable onto a top nipple on the manifold, said branch lines are inserted onto a plurality of nipples located on the manifold outer surface and a second trunk line is insertable onto a nipple located on the manifold outer surface whereby said second trunk line terminates into a connector.
